(19) United States
(12) Patent Application Publication
(10) Pub. No.: US 2023/0392198 A1
(43) Pub. Date: Dec. 7, 2023

(54) METHOD AND SYSTEM FOR DETECTING REVERSE TRANSCRIPTASE ACTIVITY BY DIGITAL ASSAY

(71) Applicant: Bio-Rad Laboratories, Inc., Hercules, CA (US)

(72) Inventors: Congcong MA, Hayward, CA (US); Duc DO, San Jose, CA (US); Douglas GREINER, Fremont, CA (US)

(73) Assignee: Bio-Rad Laboratories, Inc., Hercules, CA (US)

(21) Appl. No.: 18/324,885

(22) Filed: May 26, 2023

Related U.S. Application Data
(60) Provisional application No. 63/347,810, filed on Jun. 1, 2022.

(51) Int. Cl. C12Q 1/6844; C12N 9/12; C12Q 1/6876

(52) U.S. Cl. CPC C12Q 1/6844; C12N 9/1276; C12Q 1/6876

(57) ABSTRACT

Method and system for detecting reverse transcriptase activity of a sample by digital assay. In an exemplary method, a reaction mixture including the sample, an RNA polymer, and reagents for reverse transcription may be prepared. Complementary DNA (cDNA) may be synthesized in the reaction mixture using the RNA polymer as a template. An amount of the cDNA synthesized may be proportional to the reverse transcriptase activity of the sample. Partitions may be formed after synthesizing the cDNA. The partitions may contain copies of the cDNA at partial occupancy. Each partition may include a portion of the reaction mixture. A target representing the cDNA may be amplified in the partitions. Amplification data for the target may be collected from the partitions.

### CROSS-REFERENCE TO PRIORITY APPLICATION

This application is based upon and claims the benefit under 35 U.S.C. § 119(e) of U.S. Provisional Patent Application Ser. No. 63/347,810, filed Jun. 1, 2022. which is incorporated herein by reference in its entirety for all purposes.

### INTRODUCTION

Reverse transcriptase is an enzyme that catalyzes synthesis of complementary DNA from an RNA template in a process called reverse transcription. The enzyme is encoded by retroviruses, for which the enzyme's activity is essential in the retroviral life cycle to convert a viral RNA genome into DNA for integration into the host genome. The ability to reverse transcribe RNA into DNA has many practical applications and is required in various laboratory protocols and clinical assays. Accordingly, accurate, reliable, and safe tests for quantifying reverse transcriptase activity are important to the enzyme manufacturer for quality control, and to the end user for troubleshooting and assay verification, among others.

Known methods for quantifying reverse transcriptase activity have various drawbacks. In one approach, a relative activity is measured by testing a reverse transcriptase preparation of interest alongside a “gold standard” reverse transcriptase. However, the activity of the gold standard can be unstable and may drift over time, thereby making this approach inaccurate and unreliable. In another approach, an absolute activity of the reverse transcriptase preparation of interest is measured by quantifying incorporation of a radioactive nucleotide into the complementary DNA that is synthesized. Despite the value of measuring absolute activity, this radioactive approach is undesirable due to the licensing, inspection, cost, training, and hazards involved with storage, handling, and disposal of radioactive material.

### SUMMARY

The present disclosure provides methods and systems for detecting reverse transcriptase activity of a sample by digital assay. In an exemplary method, a reaction mixture including the sample, an RNA polymer, and reagents for reverse transcription may be prepared. Complementary DNA (cDNA) may be synthesized in the reaction mixture using the RNA polymer as a template. An amount of the cDNA synthesized may be proportional to the reverse transcriptase activity of the sample. Partitions may be formed after synthesizing the cDNA. The partitions may contain copies of the cDNA at partial occupancy. Each partition may include a portion of the reaction mixture. A target representing the cDNA may be amplified in the partitions. Amplification data for the target may be collected from the partitions.

### BRIEF DESCRIPTION OF THE DRAWINGS

FIG. 1 is a flowchart of exemplary steps that may be performed in a method of detecting reverse transcriptase activity of a sample by digital assay.

FIG. 2 is a schematic flow diagram illustrating configurations produced during performance of an exemplary method of detecting reverse transcriptase activity of a sample by digital assay.

FIG. 3 is a schematic diagram illustrating aspects of the exemplary method of FIG. 2 in more detail.

FIG. 4 is a graph schematically illustrating how varying the initial concentration of an RNA template may affect the kinetics of product (cDNA) formation by reverse transcription.

FIG. 5 is graph schematically illustrating a linear correlation between the concentration of reverse transcriptase [E] in reverse transcription reactions and the copy number of a resulting target that may be quantified in a digital assay.

FIG. 6 is a schematic diagram of nucleic acids used to test an illustrative method of detecting reverse transcriptase activity of a sample by digital assay.

FIG. 7 is a scatterplot of amplification data for a target collected from four sets of droplets formed with respective reaction mixtures containing different amounts of reverse transcriptase activity to synthesize cDNA providing the target.

FIG. 8 is a graph plotting the concentration of a target detected in replicate sets of droplets versus the concentration of a reverse transcriptase used in bulk reaction mixtures to generate cDNA providing the target.

FIG. 9 is a graph plotting the concentration of a target detected in replicate sets of droplets and produced in bulk reaction mixtures by reverse transcription with serial dilutions of each of two commercially-available reverse transcriptases.

### DETAILED DESCRIPTION

Various aspects and examples of methods, systems, and compositions for detecting reverse transcriptase activity of a sample are described below and illustrated in the associated drawings. Unless otherwise specified, the methods, systems, and compositions may, but are not required to, contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein may be included in other similar methods, systems, and compositions, including being interchangeable between disclosed examples. The following description of various examples is merely illustrative in nature and is in no way intended to limit the examples, their applications, or their uses. Additionally, the advantages provided by the examples described below are illustrative in nature and not all examples provide the same advantages or the same degree of advantage.

The present disclosure provides methods and systems for detecting reverse transcriptase activity of a sample. In an exemplary method, a reaction mixture including the sample, an RNA polymer, and reagents for reverse transcription may be prepared. Complementary DNA (cDNA) may be synthesized in the reaction mixture using the RNA polymer as a template. An amount of the cDNA synthesized may be proportional to the reverse transcriptase activity of the sample. Partitions may be formed after synthesizing the cDNA. The partitions may contain copies of the cDNA at partial occupancy. Each partition may include a portion of the reaction mixture. A target representing the cDNA may be amplified in the partitions. Amplification data for the target may be collected from the partitions.

Further aspects of the present disclosure are described in the following sections: (I) definitions, (II) overview, (Ill) examples, components, and alternatives, (IV) illustrative combinations and additional examples, (V) advantages and benefits, and (VI) conclusion.

Features, functions, and advantages may be achieved independently in various examples of the present disclosure, or may be combined in yet other examples, further details of which can be seen with reference to the following description and drawings.

### I. Definitions

Technical terms used in this disclosure have meanings that are commonly recognized by those skilled in the art. However, the following terms may be further defined as follows.

An “amplicon” is a product of an amplification reaction. Copies of an amplicon may be generated by amplification of a target sequence, such that the amplicon corresponds to the target sequence (i.e., matches and/or is complementary to the target sequence). However, the sequence of the amplicon, such as at primer binding sites, may not exactly match and/or may not be perfectly complementary to the target sequence.

“Amplification” is a process whereby multiple copies are made of an amplicon matching, complementary, and/or otherwise corresponding to a target sequence. The process interchangeably may be called an amplification reaction. Amplification may generate an exponential increase in the number of copies as amplification proceeds. Typical amplifications may produce a greater than 100-fold or 1,000-fold increase in the number of copies of an amplicon. Exemplary amplification reactions for the methods disclosed herein may include a polymerase chain reaction (PCR) or a ligase chain reaction (LCR), each of which is driven by thermal cycling. The methods also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and/or the like. Amplification may utilize a linear or circular template.

“Amplification reagents” are any reagents that promote generation of an amplicon by amplification of a target sequence. The reagents may include any combination of at least one primer or primer pair for amplification of at least one target sequence, at least one label for detecting amplification of the at least one target sequence (e.g., at least one probe including a fluorophore and/or an intercalating dye as a label), at least one polymerase enzyme and/or ligase enzyme (which may be heat-stable), and nucleoside triphosphates (dNTPs and/or NTPs), among others.

A “carrier fluid” is a fluid that contacts partitions, optionally enclosing each partition. The fluid may be liquid or gas. The carrier fluid may be described as a continuous phase and the partitions therein as a dispersed phase. The carrier fluid may be immiscible with, and encapsulate each partition. In some examples, the carrier fluid may be an oil, such as including a fluorocarbon oil or a silicone oil.

“Complementary” means related by the rules of base pairing. A first nucleic acid polymer, or region thereof, is “complementary” to a second nucleic acid polymer if the first nucleic acid polymer or region is capable of hybridizing with the second nucleic acid polymer in an antiparallel fashion by forming a consecutive (uninterrupted) or nearly consecutive series of base pairs (e.g., at least 5, 6, 7, 8, 9, or 10 consecutive base pairs). The first nucleic acid polymer (or region thereof) is termed “perfectly complementary” to the second nucleic acid polymer if hybridization of the first nucleic acid (or region thereof) to the second nucleic acid polymer forms a consecutive series of base pairs using every nucleotide of the first nucleic acid polymer or region thereof. A “complement” of a first nucleic acid polymer or region thereof is a second nucleic acid polymer or region thereof that is perfectly complementary to the first nucleic acid polymer or region thereof. The “complementarity” between a first nucleic acid polymer (or region thereof) and a second nucleic acid polymer (or region thereof) refers to the number or percentage of base pairs that can be formed when the first nucleic acid polymer (or region thereof) is optimally aligned for hybridization in an antiparallel fashion with the second nucleic acid polymer (or region thereof). A first nucleic acid polymer or region thereof that is complementary to a second nucleic acid polymer or region thereof generally has a complementarity of at least 80%, 90%, 95%, or 100%.

“Complementary DNA” (cDNA) is a DNA polymer that is complementary to a given RNA polymer, where the RNA polymer serves as a template for synthesis of the DNA polymer, generally in the presence of reverse transcriptase.

“Comprising,” “including,” and “having” (and conjugations thereof) are used interchangeably to mean including but not necessarily limited to, and are open-ended terms not intended to exclude additional, unrecited elements or method steps.

A “digital assay” is an investigative procedure(s) capable of detecting single copies of an analyte, such as a nucleic acid target, in a set of subsamples or partitions, in which each subsample/partition of only a subset of the subsamples/partitions contains one or more copies of the analyte. A “digital amplification assay” is a digital assay that utilizes an amplification reaction(s) to facilitate detection of single copies of a target(s). A digital assay may be performed with any suitable number of subsamples/partitions that gives a statistically significant result, such as at least twenty, one-hundred, one-thousand, or ten-thousand, among others.

A “droplet” is a small volume of liquid encapsulated by an immiscible fluid (e.g., encapsulated by an immiscible liquid, which may form a continuous phase of an emulsion). The immiscible liquid may include oil and/or may be composed predominantly of oil. Droplets disclosed herein may, for example, have an average volume of less than about 500 nL, 100 nL, 10 nL, or 1 nL, among others.

An “enumeration value” is any value that results from enumerating a target-defined subset of a set of subsamples/partitions in a digital assay. The enumeration value may, for example, represent a number of subsamples/partitions in the set of subsamples/partitions that are positive for the presence of a given target or two or more given targets, negative for the presence of a given target or two or more given targets, positive for only a specified subset of one or more targets of a set of targets, negative for only a specified subset of one or more targets of a set of targets, or the like.

“Exemplary” means “illustrative” or “serving as an example.” Similarly, the term “exemplify” (or “exemplified”) means “to illustrate by giving an example.” Neither term implies desirability or superiority.

“First,” “second,” “alpha,” “beta,” and similar terms are used to distinguish or identify various members of a group, or the like, in the order they are introduced in a particular context and are not intended to show serial or numerical limitation.

“Fluorescence” is optical radiation emitted in response to absorption of light. As used herein, fluorescence is intended to cover any form of photoluminescence, in which absorption of one or more photons promotes an electron to an excited state and leads to subsequent emission of a new photon, whether from a singlet state, a triplet state, or other state. The excited state produced by absorption may have any suitable lifetime.

A “fluorophore” is any atom, functional group, moiety, or substance capable of fluorescence.

A “label” is any detectable marker or identifier associated with a member, structure, or substance, such as associated with a primer, probe, amplicon, subsample, partition, or the like. The label may be associated covalently with the member, structure, or substance, such as a label that is conjugated to an oligonucleotide or antibody, or associated non-covalently (e.g., by intercalation, hydrogen bonding, electrostatic interaction, encapsulation, etc.). Exemplary labels include optical labels, radioactive labels, magnetic labels, electrical labels, epitopes, enzymes, antibodies, oligonucleotides, etc. Optical labels are detectable optically via their interaction with light. Exemplary optical labels that may be suitable include fluorophores and quenchers, among others.

“Labeling” is any action, process, or procedure that connects or associates a label to or with a specified structure, member, or substance. Labeling may produce a non-covalent connection or association, or a covalent attachment, of the label to the structure, member, or substance.

A “nucleic acid polymer” is a molecule or molecular duplex of any length composed of naturally-occurring nucleotides (e.g., where the polymer is an RNA polymer (also called RNA) or a DNA polymer (also called DNA)), or a compound produced synthetically that can hybridize with DNA or RNA in a sequence-specific manner analogous to that of two naturally occurring nucleic acids, e.g., can participate in Watson-Crick base pairing interactions. A nucleic acid polymer may be composed of any suitable number of nucleotides, such as at least about 5, 10, 100, or 1000, among others. The term “nucleic acid” means one or more nucleic acid polymers.

A nucleic acid polymer may have a natural or artificial structure, or a combination thereof. Nucleic acid polymers with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (G)) or a pyrimidine (such as cytosine (C), thymine (T), or uracil (U))). Nucleic acid polymers with an artificial structure are analogs of natural nucleic acids and may, for example, be created by changes to the pentose and/or phosphate groups of the natural backbone and/or to one or more nucleobases. Exemplary artificial nucleic acid polymers include glycol nucleic acids (GNAs), peptide nucleic acids (PNAs), locked nucleic acids (LNAs), threose nucleic acids (TNAs), xeno nucleic acids (XNA), and the like.

The sequence of a nucleic acid polymer is defined by the order in which nucleobases are arranged along the backbone. This sequence generally determines the ability of the nucleic acid polymer to hybridize with another nucleic acid by hydrogen bonding. In particular, adenine pairs with thymine (or uracil) and guanine pairs with cytosine.

An “oligonucleotide” is a relatively short and/or chemically synthesized nucleic acid polymer. The length of an oligonucleotide may, for example, be 5 to 1000 nucleotides, among others. In some cases, an oligonucleotide may be labeled with at least one label, which may be conjugated to any suitable structure of the oligonucleotide. The at least one label may include at least one fluorophore and thus may be a fluorescent label. Each label may be conjugated to the oligonucleotide at any suitable position, including a 5′-end, a 3′-end, or intermediate the 5′- and 3′-ends.

“Optical radiation” means electromagnetic radiation in the optical spectrum, namely, ultraviolet light, visible light, and/or infrared light. Accordingly, the term “light” has the same meaning as optical radiation.

“Partitions” are discrete volumes of fluid (i.e., fluid volumes) that are spatially-isolated from one another (also called isolated volumes). Each partition of a set of partitions may contain a portion of the same sample. The partitions may be separated from one another by fluid (e.g., oil or air), a wall(s) of a device(s), or a combination thereof, among others. Accordingly, the partitions may, for example, be droplets of an emulsion, or volumes held by separate fluid-holding structures, such as wells (e.g., microwells each having a capacity of less than 1 μL), chambers (e.g., microchambers each having a capacity of less than 1 μL), tubes (e.g., microtubes each having a diameter of less than 1 mm), or microfluidic devices, among others. The partitions may have the same diameter as one another and/or may be composed of the same amount of fluid as one another.

“Partial occupancy” means present or contained in only a subset of a set of partitions. A set of partitions containing a target at partial occupancy means that at least one copy of the target is present in each partition of only a subset of the partitions. In other words, one or more of the partitions do not contain any copies of the target. Copies of the target may be distributed randomly among the partitions but may be in limited supply such that every partition fails to receive a copy of the target.

A “partition count” (or a “subsample count”) is a value for the number of partitions (or subsamples) in a specified group, such as a population of partitions (or subsamples) having a specified target content. A partition count or subsample count may be an enumeration value. The partition/subsample count may be determined by counting, estimating, and/or calculating.

The term “positive” when used to indicate a target content of a partition, subsample, or a population of partitions/subsamples indicates that the partition/subsample, or each partition/subsample of the population contains (or at least appears and/or is deemed to contain) at least one copy of a given target or of each target of a given set of targets. The term “negative” when used to indicate a target content of a partition/subsample or population of partitions/subsamples indicates that the partition/subsample, or each partition/subsample of the population does not contain (or at least appears and/or is deemed not to contain) at least one copy of a given target or of each target of a given set of two or more targets.

A “primer” is an oligonucleotide (DNA/RNA or an analog thereof) capable of serving as a point of initiation of template-directed nucleic acid synthesis or ligation under appropriate reaction conditions (e.g., in the presence of a template to which the primer anneals, nucleoside triphosphates, and an agent for polymerization (such as a DNA or RNA polymerase or ligase, or a reverse transcriptase), in an appropriate buffer and at a suitable temperature). The primer may have any suitable length, such as 5 to 500 nucleotides, among others. The primer may be a member of a “primer pair” or “primer set” including a “forward primer” and a “reverse primer” that define the ends of an amplicon generated in an amplification reaction. (The adjectives “forward” and “reverse” are arbitrary designations relative to one another.) The forward primer hybridizes with a complement of the 5′-end region of a template sequence to be amplified, and the reverse primer hybridizes with the 3′-end region of the template sequence. The term “primer binding site” refers to a portion of a template (or its complement) to which a primer anneals. The full sequence of the primer need not be perfectly complementary to the primer binding site, just sufficiently complementary to anneal under the conditions of the reaction. Accordingly, the primer may have a 3′-end region that is complementary to the primer binding site, and a 5′-end region that is not complementary to the primer binding site (and forms a “5′-tail”).

A “probe” is any substance configured to facilitate or enable testing, identification, and/or detection of an analyte. The probe may, for example, be an amplification probe, which may include an oligonucleotide. An amplification probe is configured to enable detection of the occurrence of an amplification reaction and/or formation of an amplicon by the amplification reaction. In some examples, the amplification probe may be a fluorescent probe including an oligonucleotide labeled with a fluorophore. The amplification probe may be configured to hybridize with at least a portion of an amplicon generated by amplification. The amplification probe may, for example, be a hydrolysis probe, a molecular beacon probe, a strand displacement probe, or a labeled primer, among others.

A “reverse transcriptase” is an enzyme that catalyzes “reverse transcription,” which is the synthesis of complementary DNA (cDNA) using an existing RNA polymer as a template. The cDNA may be synthesized by extending a primer that is hybridized with the RNA polymer. This extension may utilize one or more deoxyribonucleoside triphosphates (dNTPs) as substrates for extension of the primer.

“Reverse transcriptase activity” is the capacity of a sample, stock solution, and/or enzyme preparation containing reverse transcriptase to catalyze reverse transcription. This activity may be defined using any suitable activity units. For example, a unit may be defined as the amount of enzyme needed to catalyze synthesis of a specified quantity of cDNA, incorporation of a specified quantity of nucleotides into cDNA, or the like, under defined reaction conditions (e.g., temperature, reaction duration, RNA template sequence(s) and concentration, primer sequence(s) and concentration, dNTP(s) concentration, buffer composition, etc.). The activity may be defined per unit volume, per enzyme mass, per enzyme number/moles, per total protein mass, or the like.

A “subsample” is a smaller sample provided by a bulk sample or sample-containing fluid and containing only a portion of the bulk sample or sample-containing fluid. A set of subsamples may be a set of partitions, and vice versa.

“Substantially” means to be predominantly conforming to the particular dimension, range, shape, concept, or other aspect modified by the term, such that a feature or component need not conform exactly, so long as it is suitable for its intended purpose or function. For example, a “substantially cylindrical” object means that the object resembles a cylinder, but may have one or more deviations from a true cylinder.

A “target” (also called a “target sequence”) is a nucleic acid polymer sequence (DNA and/or RNA) of any suitable length that is amplified in an amplification reaction. Exemplary target sequences are about 20-1000 nucleotides, or about 30-500 nucleotides, among others.

A “target-defined subset” of a set of partitions is composed of partitions having a defined target content (positivity/negativity) of one or more targets.

A “template” is a nucleic acid polymer (e.g., an RNA or DNA polymer) that serves as a pattern for the generation of another nucleic acid polymer.

### II. Overview

This section provides an overview of the methods and systems described herein.

A method of detecting reverse transcriptase activity of a sample is provided. In the method, a reaction mixture including the sample, an RNA polymer, and reagents for reverse transcription may be prepared. Complementary DNA (cDNA) may be synthesized in the reaction mixture using the RNA polymer as a template. An amount of the cDNA synthesized may be proportional to the reverse transcriptase activity of the sample. Partitions may be formed after synthesizing the cDNA. The partitions may contain copies of the cDNA at partial occupancy. Each partition may include a portion of the reaction mixture. A target representing the cDNA may be amplified in the partitions. Amplification data for the target may be collected from the partitions.

A system for measuring reverse transcriptase activity of a sample is provided. The system may comprise a plurality of partitions containing a cDNA at partial occupancy. Each partition may include a portion of a reaction mixture in which the cDNA was synthesized by reverse transcription using the reverse transcriptase activity of the sample and an RNA polymer as a template. The cDNA may be present in an amount that is proportional to the reverse transcriptase activity of the sample. Each partition also may include reagents sufficient for amplification of a target representing the cDNA, if a copy of the cDNA is present in the partition.

### III. Examples, Components, and Alternatives

The following subsections, A to D, describe selected aspects of exemplary methods and systems for detecting reverse transcriptase activity of a sample by digital assay. The examples in these subsections are intended for illustration and should not be interpreted as limiting the entire scope of the present disclosure. Each subsection may include one or more distinct examples, and/or contextual or related information, function, structure, and/or processes.

### A. Methods of Detecting Reverse Transcriptase Activity

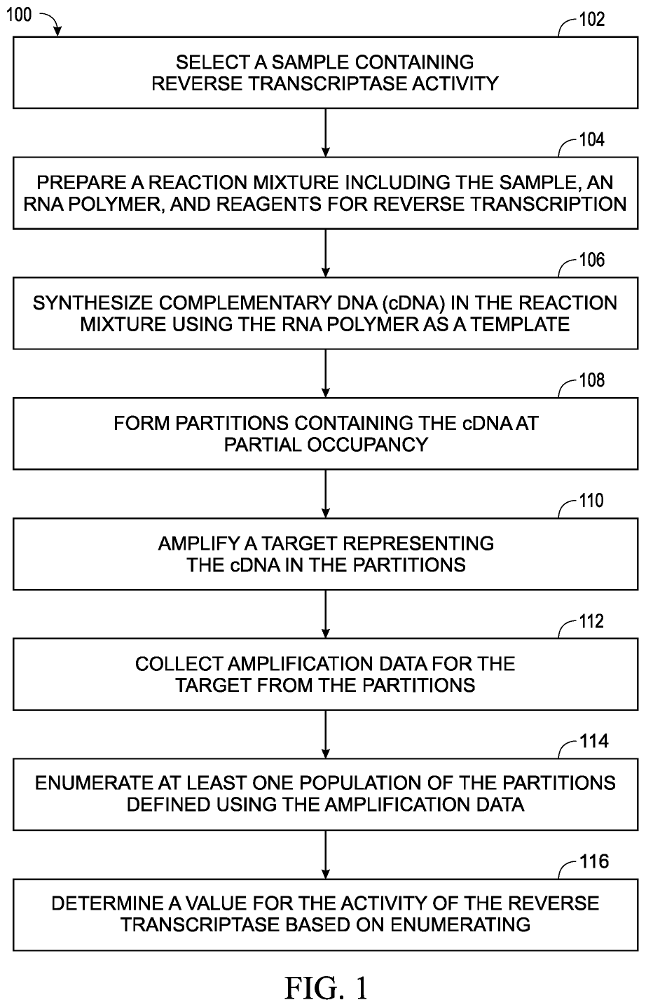

This subsection describes illustrative methods of detecting reverse transcriptase assay using a digital assay; see FIG. 1. Each method may include any suitable combination of the steps listed in flowchart 100 of FIG. 1, performed in any suitable order, and with any of the steps omitted or performed any suitable number of times, and using or including any of the features, aspects, configurations, additional steps, or modifications described elsewhere in the present disclosure.

In a selecting step 102, a sample containing reverse transcriptase activity is selected. The sample may contain a reverse transcriptase that has been purified, expressed from an engineered construct, modified to change its primary amino acid sequence relative to a natural reverse transcriptase (e.g., to eliminate RNAse H activity, increase thermal stability, alter fidelity, and/or the like), or any combination thereof. In other cases, the sample may contain a reverse transcriptase that has been expressed in an infected cell(s).

In a preparing step 104, a reaction mixture including the sample, an RNA polymer, at least one reverse transcription (RT) primer, and reagents for reverse transcription may be prepared. The RNA polymer may have a predefined sequence or may include two or more RNA molecules having different sequences. The RNA polymer may be expressed from an engineered construct, and/or may be purified. Each RT primer independently may have a single nucleotide sequence or may be a degenerate primer. The RT primer may be complementary to the RNA polymer and configured to hybridize with the RNA polymer, at a position spaced from the 5′-end of the RNA polymer (e.g., closer to the 3′-end of the RNA polymer than the 5′-end). The reagents may include one or more dNTPs (e.g., dATP, dCTP, dGTP, and/or dTTP) according to nucleotide requirements of the cDNA to be synthesized.

The reaction mixture also may be prepared at this stage to contain all of the amplification reagents sufficient for later target amplification. Accordingly, the reaction mixture may include at least one amplification primer or at least one pair of amplification primers for target amplification, and an enzyme to catalyze amplification. The enzyme may be a DNA polymerase (e.g., a heat-stable polymerase) to extend the amplification primer(s), or a DNA ligase (e.g., a heat-stable DNA ligase) to ligate amplification primers to one another. In other examples, one or more of the amplification reagents may be added to the reaction mixture after performing reverse transcription.

In a synthesizing step 106, complementary DNA (cDNA) may be synthesized in the reaction mixture using the RNA polymer as a template. Synthesizing step 106 may be performed at a controlled temperature(s), and may be started and stopped by changing the temperature of the reaction mixture. For example, synthesis may be started by heating the reaction mixture to a first predefined temperature, and synthesis may be stopped by heating or cooling the reaction mixture to a second predefined temperature. In other examples, synthesizing step 106 may be started by addition of a reagent(s) for reverse transcription, and/or may be stopped by addition of an inhibitor of reverse transcription.

In a forming step 108, partitions containing the cDNA at partial occupancy may be formed. Each partition contains a portion of the reaction mixture. Each partition of only a subset of the partitions contains at least one copy of the cDNA. Forming step 108 may include dividing the reaction mixture into spatially-isolated fluid volumes. The spatially isolated-fluid volumes may be separated from one another by an immiscible fluid (e.g., when the volumes are droplets or slugs), or each may be contained by a separate fluid-holding structure (e.g., a well (such as a microwell), a chamber (such as a microchamber), or the like.

In an amplifying step 110, a target representing the cDNA may be amplified in the partitions. The target may be provided by the cDNA and may correspond to any suitable portion of the cDNA. Amplifying step 110 may include thermally cycling the partitions through a plurality of cycles. Each cycle may include a denaturation phase, an annealing phase, and an extension phase, which be achieved by heating the partitions to a denaturation temperature, cooling the partitions to an annealing temperature, and heating the partitions to an extension/ligation temperature. In other examples, the annealing temperature and the extension/ligation temperature may be the same as one another.

In a collecting step 112, amplification data for the target may be collected from the partitions. Collecting step 112 may be performed after amplifying step 110 has been completed. The amplification data may be collected by detecting light, such as fluorescence, from the partitions. The intensity of fluorescence detected from each partition may be dependent on whether the target was amplified in the partition.

In an enumerating step 114, at least one population of the partitions may be enumerated, where the at least one population is defined using the amplification data. A signal detected from each partition in the collecting step may be compared to a threshold, to classify the partition as positive or negative for the target. A positive or negative partition count for partitions that are negative for the target, or that are positive for the target, may be obtained. A total partition count also may be obtained. A fraction of the partitions that are negative for the target, or that are positive for the target, may be calculated using the positive or negative partition count and the total partition count. An average target concentration per partition may be calculated based on the fraction, e.g., with Poisson statistics. A target concentration per unit volume may be calculated based on the average volume or total volume of the partitions.

In a determining step 116, a value for the reverse transcriptase activity of the sample may be determined based on enumerating. Since each target represents one molecule of the cDNA, the target concentration obtained above may be utilized to calculate an amount of cDNA synthesized in the reverse transcription reaction by the amount of reverse transcriptase present in the sample. Since there is a linear correlation between the amount of enzyme and the amount of target/cDNA synthesized, the amount of target/DNA synthesized can be used to determine absolute enzyme activity without a reference reverse transcriptase.

### B. Illustrative Flow Diagram

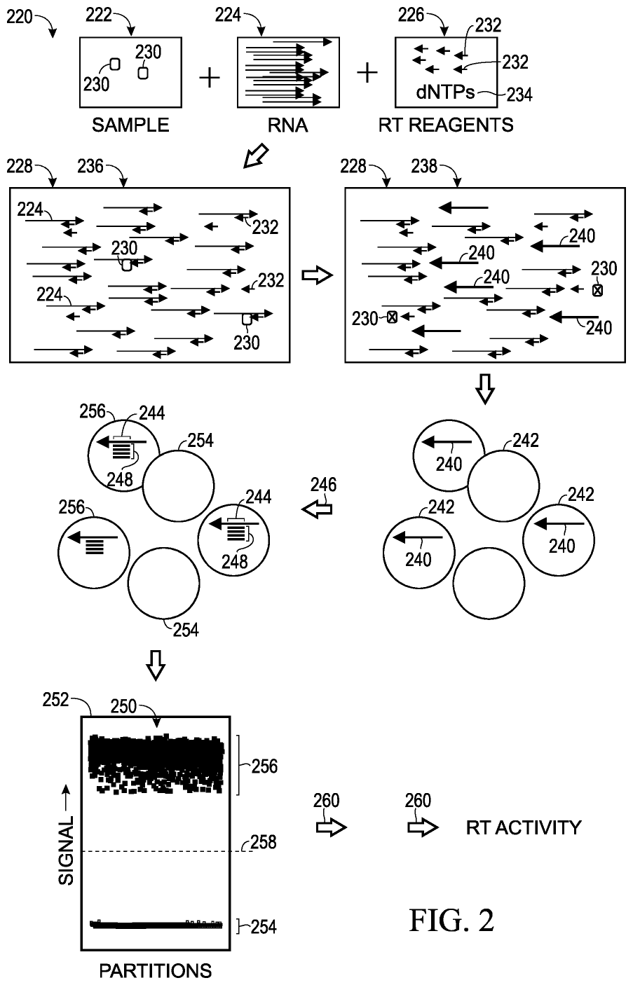

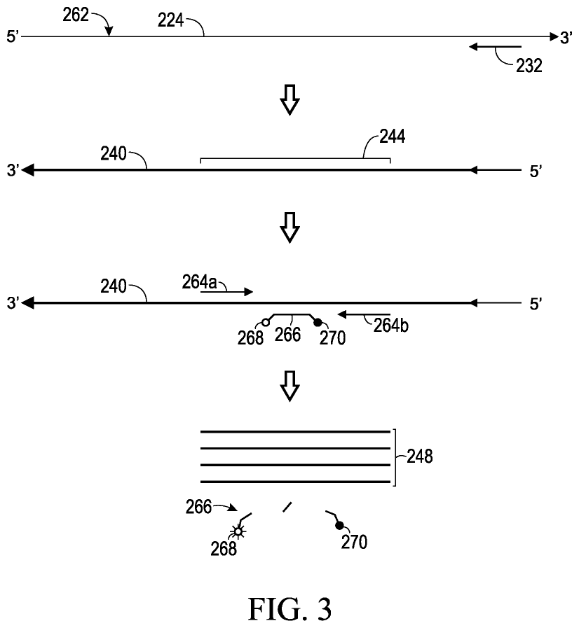

This subsection describes a schematic flow diagram 220 illustrating configurations produced during performance of an exemplary method of detecting reverse transcriptase activity of a sample 222 by digital assay; see FIGS. 2 and 3. Further exemplary aspects of the method are described above in subsection A.

Sample 222 may be combined with an RNA polymer 224 and reagents 226 for reverse transcription (RT), to prepare a reaction mixture 228 (see FIG. 2). The components of reaction mixture 228 may be combined with one another in any suitable order. For example, the RNA polymer and the RT reagents may be present together in a preformed composition, which is combined with the sample.

Sample 222 may contain copies of at least one type of reverse transcriptase 230. The sample may be a commercial/laboratory sample containing a commercially-produced and/or engineered reverse transcriptase, which may be expressed from an engineered construct and/or purified. In other cases, the sample may be a clinical sample, an environmental sample, or the like, which may or may not contain a reverse transcriptase expressed naturally.

RNA polymer 224 may be predominantly or exclusively composed of copies of the same RNA sequence. The RNA polymer may be expressed from an engineered construct and/or may be purified.

RT reagents 226 may include copies of at least one RT primer 232 and dNTPs 234 (deoxyribonucleoside triphosphates) for extension of the RT primer catalyzed by reverse transcriptase 230. Each RT primer 232 is configured to anneal to RNA polymer 224 by base-pairing, such that extension of the primer uses the RNA polymer as a template.

Reaction mixture 228 contains a substantial excess of RNA polymer 224 copies over reverse transcriptase 230 copies. The excess of RNA polymer 224 (and RT reagents 226) ensures that the reverse transcriptase remains rate-limiting for reverse transcription throughout the duration of the reaction, until the reaction is stopped. The molar ratio of RNA polymer 224 to reverse transcriptase 230, when reaction mixture 228 is first formed, may be at least 10:1, 50:1, 100:1, 500:1, or 1000:1, among others. RT primer 232 may be present in an equal amount or a molar excess over RNA polymer 224, as shown.

Reaction mixture 228 is shown in an initial, unreacted configuration 236 on the left and in a final, reacted configuration 238 on the right after the reverse transcription reaction has been stopped. Incubation of the reaction mixture at a defined temperature(s) for a defined time interval allows reverse transcriptase 230 to catalyze synthesis of cDNA 240 by extension of RT primer 232 with RNA polymer 224 as a template. In this highly simplified example, each copy of reverse transcriptase 230 has catalyzed only three cycles of cDNA synthesis, but in other examples, each active copy of reverse transcriptase 230 may be responsible for synthesis of an average of at least 10, 50, 100, 500, or 1000 copies of cDNA 240. The cDNA may be single stranded, as shown, or may be hybridized with RNA in an RNA/DNA hybrid or with itself intramolecularly to create a stem-loop structure, among others.

The reverse transcription reaction may be stopped at the end of the defined time interval by inactivating reverse transcriptase 230 (indicated by an “X” in each copy of the reverse transcriptase). In some examples, inactivation may be performed by heating the reaction mixture to an inactivating temperature above the defined reaction temperature, to denature the reverse transcriptase. The inactivating temperature may, for example, be at least 60, 70, or 80 degrees.

Partitions 242 each containing a portion of reaction mixture 228 may be formed. The partitions contain cDNA 240 at partial occupancy, such that only a subset of partitions 242 contain a copy of the cDNA, as shown. Each partition may contain the same volume of the reaction mixture. Each partition also contains reagents sufficient for amplification of a target 244 from cDNA 240 in partitions 242, indicated by an amplification arrow 246, to generate an amplicon 248.

Amplification data 250 may be collected from partitions 242, as presented in a plot 252 of a signal amplitude (e.g., fluorescence intensity) detected from each individual partition 242. In the present example, negative partitions 254 (no target amplification) have a weaker signal, and positive partitions 256 with target amplification have a stronger signal. The signal from each partition may be compared with a threshold 258 to assign the partition as negative or positive.

The amplification data may be processed, indicated by processing arrows 260, to determine a reverse transcriptase activity of the sample. For example, negative partitions 254 or positive partitions 256, and total partitions 242 (positive and negative) may be enumerated to obtain enumeration values (partition counts). The reverse transcriptase activity may be defined according to any suitable convention. For example, one or more of the enumeration values may be utilized to calculate a value for the average number of target copies (and/or cDNA copies) present per partition and/or unit volume, and/or a value for the total number of target copies (and/or cDNA copies) present in the reaction mixture or per unit volume of the reaction mixture or sample.

FIG. 3 shows further illustrative aspects of the exemplary method of FIG. 2 in more detail. A hybrid 262 of a copy of RT primer 232 annealed to a copy of RNA polymer 224 is shown at the top. A copy of cDNA 240 produced by extension of RT primer 232 is shown below hybrid 262. A pair of amplification primers 264a, 264b and a hydrolysis probe 266 are shown positioned along the copy of cDNA where each hybridizes to the cDNA (or to a complement thereof). Copies of amplicon 248 generated by amplification of target 244 with primers 264a, 264b are shown below the primers, along with a degraded form of hydrolysis probe 266. The hydrolysis probe includes a fluorophore 268 and a quencher 270. Degradation of the hydrolysis probe as a result of target amplification increases the fluorescence of fluorophore 268, because quencher 270 is no longer tethered in proximity to the fluorophore.

### C. Illustrative Kinetic Relationships in Reverse Transcription Assays

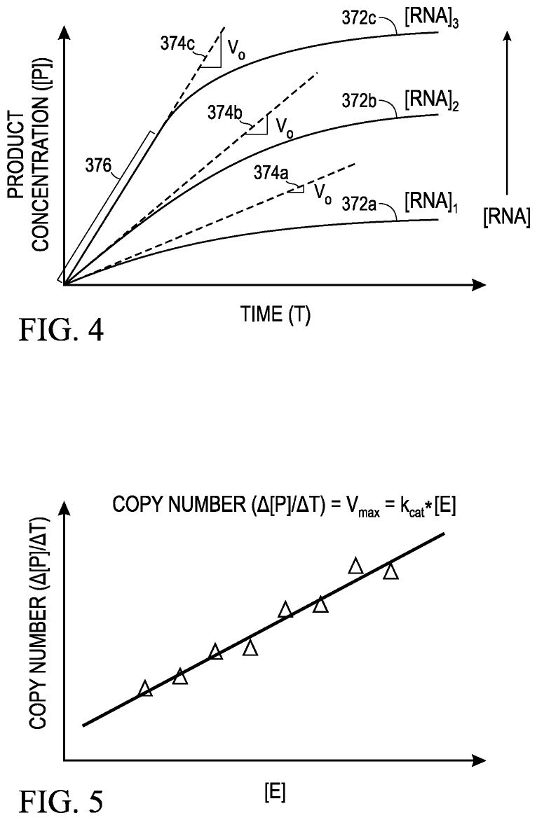

This subsection describes kinetic relationships between concentrations of a substrate (an RNA polymer), a product (a cDNA synthesized using the RNA polymer as a template), and an enzyme (a reverse transcriptase) in reverse transcription assays; see FIGS. 4 and 5, which are schematic in nature and do not present actual data.

FIG. 4 illustrates how varying the initial concentration of an RNA polymer (the template), while keeping the concentration of reverse transcriptase constant, may affect the kinetics of product (cDNA) formation catalyzed by the reverse transcriptase. Product accumulation curves 372a, 372b, and 372c are presented for three reverse transcription reactions performed with respectively-increasing initial concentrations of an RNA polymer [RNA]₁, [RNA]₂, and [RNA]₃). The initial rate of each reaction, Vo, is indicated as the slope of respective velocity lines 374a, 374b, and 374c that are tangential to the corresponding accumulation curves and intersect the origin. At the two lower concentrations of the RNA polymer, [RNA]₁ and [RNA]₂, the enzyme is not saturated with substrate at the start of the reaction, so that Vo is less than the maximum rate, Vₘₐₓ, and product accumulation curves 372a and 372b diverge from velocity lines 374a and 374b early in the reaction. In contrast, at the highest concentration of the RNA polymer, [RNA]₃, the reaction begins with the enzyme saturated with substrate, and the enzyme remains saturated with substrate during a linear phase of the reaction producing a linear section 376 of accumulation curve 372c. During the linear phase, Vo is equal to Vₘₐₓ. The reverse transcription (RT) reactions disclosed herein may be conducted with a concentration of RNA polymer sufficient to keep the reverse transcriptase saturated with the RNA polymer from the start to the end of each RT reaction. Accordingly, the RT reaction remains at Vₘₐₓ and the product accumulates linearly during the entire reaction.

Equation 1 defines a relationship between Vₘₐₓ and the total concentration of active enzyme [Eₜ] (i.e., the number of active enzyme sites per unit volume):

Vₘₐₓ=k_{cat}[Eₜ] (1)

where k_{cat} is the catalytic constant or turnover number, which is a measure of the number of molecules of substrate converted to product per unit time (e.g., per second) per active site of the enzyme. Since k_{cat} is constant for a given enzyme and set of reaction conditions (i.e., temperature, buffer, pH, etc.), Vₘₐₓ is proportional to the total concentration of active enzyme under those reaction conditions. Therefore, a value measured for Vₘₐₓ can serve as a proxy for the concentration of active reverse transcriptase, whether or not the value of k_{cat} is known.

FIG. 5 shows a graph illustrating a linear relationship between the concentration of a reverse transcriptase (E) present in an RT reaction and the copy number of a cDNA product (P) synthesized in the RT reaction per unit volume and unit time. Each RT reaction is indicated by a triangle in the graph. The copy number of the cDNA per unit volume and time is equal to the Vₘₐₓ of the RT reaction and can be measured in a digital assay by amplification of a target present in the cDNA.

### D. Exemplary Test Results

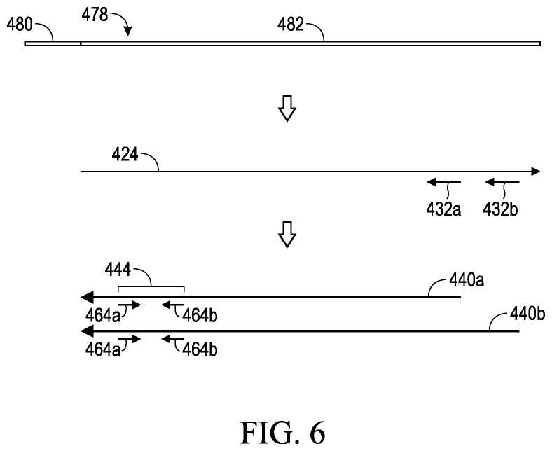

This subsection describes exemplary test results obtained by detecting reverse transcription activity using a digital assay; see FIGS. 6-9.

FIG. 6 shows a schematic diagram of nucleic acid polymers that were constructed or synthesized. A 1.5 kb expression construct 478 was amplified from a plasmid, purified, and analyzed on a Bioanalyzer High Sensitivity DNA chip (Agilent Technologies). Expression construct 478 has a T7 RNA polymerase promoter sequence 480 fused to an artificial (“alien”) template sequence 482. The artificial template sequence was transcribed in vitro by T7 RNA polymerase into a 1.5 kb alien RNA polymer 424, which then was purified by DNAse I digestion and lithium chloride precipitation.

RT reactions including RNA polymer 424 as a template were performed as follows. RT reaction mixtures were prepared on ice in a 96-well PCR plate. Each reaction mixture contained 1x RT buffer (50 mM Tris pH 8.3, 75 mM KCl, 3 mM MgCl₂); 0.5 U/4 RNase Inhibitor (Murine, New England Biolabs); RT primers 432a and 432b, each at 1 μM; dNTPs, each at 1 mM; RNA polymer 424 at 50 nM; and iScript™ Advanced reverse transcriptase (Bio-Rad) at 0.8-1.4 nM. The PCR plate was transferred to a thermal cycler and incubated at 4° C. for 30 minutes, 39° C. for 20 minutes, 85° C. for 10 minutes, and held at 4° C. cDNA 440a, 440b was synthesized by extension of respective RT primers 432a, 432b. Each RT reaction was assayed in a set of droplets by digital PCR using a pair of amplification primers 464a, 464b to amplify a target 444 present in cDNA 440a, 440b, and a fluorescent probe.

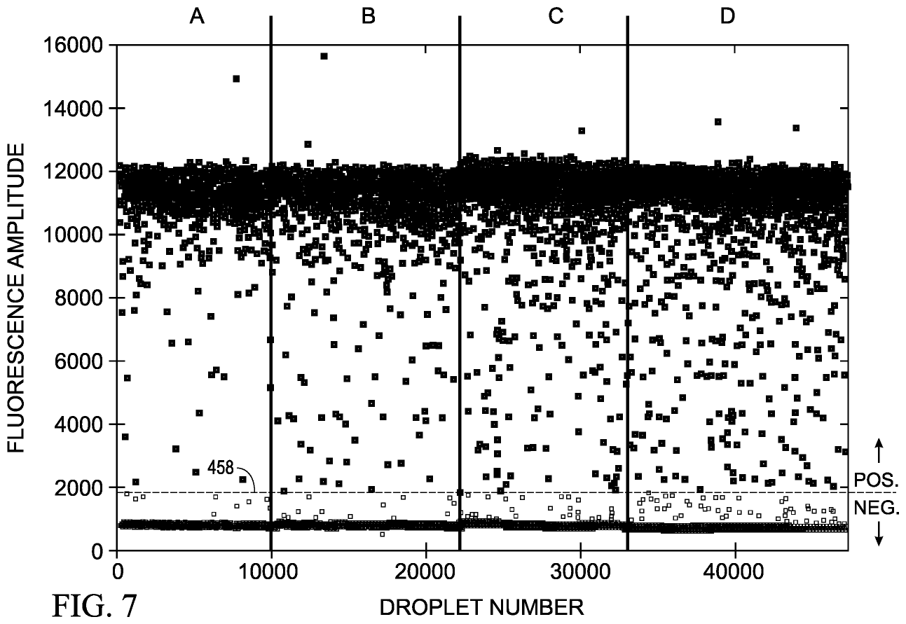

FIG. 7 shows a scatterplot of amplification data collected from four sets of droplets (A-D) containing cDNA 440a, 440b synthesized with different, respectively-increasing concentrations of reverse transcriptase (also see FIG. 6). Droplets deemed positive for the cDNA (440a and/or 440b) have a fluorescence amplitude above a threshold 458, and those deemed negative for the cDNA have a fluorescence amplitude below the threshold.

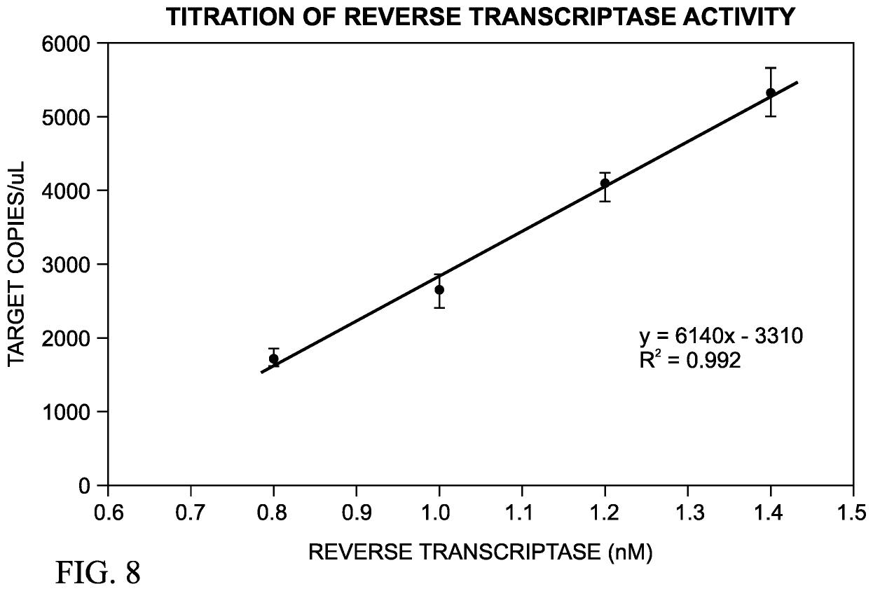

FIG. 8 shows a graph plotting the concentration (copies/μL) of target 444 measured from sets of replicate RT reactions versus the concentration of iScript™ Advanced reverse transcriptase used to generate cDNA 440a, 440b in each set of replicate RT reactions (also see FIG. 6). The error bars represent standard deviation for each set of replicates. The coefficient of determination (R²) is 0.992, and the coefficient of variation is less than 10%. The assay can detect a 20% difference in reverse transcriptase activity/concentration.

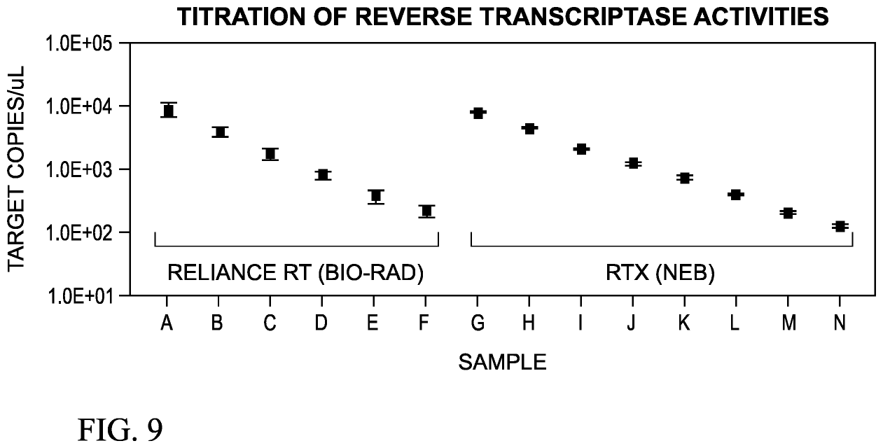

FIG. 9 shows a graph plotting the concentration of target 444 amplified from cDNA 440a, 440b synthesized as described above, except that two other commercially-available reverse transcriptases were used to catalyze the reverse transcription reactions (also see FIG. 6). Sample sets A-F are serial two-fold dilutions of Reliance RT (Bio-Rad), and sample sets G-N are serial two-fold dilutions of RTX (New England Biolabs).

### IV. Illustrative Combinations and Additional Examples

This section describes additional aspects and features of the methods and systems of the present disclosure, presented without limitation as a series of paragraphs, some or all of which may be alphanumerically indexed for clarity and efficiency. Each of these paragraphs can be combined with one or more other paragraphs, and/or with disclosure from elsewhere in this application, in any suitable manner. Some of the paragraphs below expressly refer to and further limit other paragraphs, providing without limitation examples of some of the suitable combinations.

A1. A method of detecting reverse transcriptase activity of a sample, the method comprising: preparing a reaction mixture including the sample, an RNA polymer, and reagents for reverse transcription; synthesizing complementary DNA (cDNA) in the reaction mixture using the RNA polymer as a template, wherein an amount of the cDNA synthesized is proportional to the reverse transcriptase activity of the sample; forming partitions after synthesizing, the partitions containing copies of the cDNA at partial occupancy and each partition including a portion of the reaction mixture; amplifying a target representing the cDNA in the partitions; and collecting amplification data for the target from the partitions.

A2. The method of paragraph A1, further comprising enumerating at least one population of the partitions defined according to positivity or negativity for the target using the amplification data.

A3. The method of paragraph A2, further comprising enumerating the partitions without respect to positivity/negativity for the target to obtain a total number of the partitions.

A4. The method of paragraph A2 or A3, further comprising determining a value for the reverse transcriptase activity based on enumerating.

A5. The method of paragraph A4, wherein determining includes calculating a fraction of the partitions that is positive for the target or a fraction of the partitions that is negative for the target.

A6. The method of paragraph A4 or A5, wherein determining includes calculating an average number of molecules of the cDNA per partition or unit volume.

A7. The method of any of paragraphs A1 to A6, wherein preparing the reaction mixture includes combining the sample and the RNA polymer with one another.

A8. The method of any of paragraphs A1 to A7, wherein the RNA polymer is a product of transcription in vitro.

A9. The method of any of paragraphs A1 to A8, wherein the sample includes at least one reverse transcriptase that produces the reverse transcriptase activity, and wherein the RNA polymer is present in at least a 20-fold, 50-fold, or 100-fold molar excess relative to the at least one reverse transcriptase after preparing the reaction mixture.

A10. The method of any of paragraphs A1 to A9, wherein the reagents for reverse transcription include deoxyribonucleoside triphosphates (dNTPs) and at least one primer for cDNA synthesis with the RNA polymer as template.

A11. The method of any of paragraphs A1 to A10, wherein the sample includes at least one reverse transcriptase that produces the reverse transcriptase activity, and wherein the RNA polymer is present at a non-limiting concentration with respect to the at least one reverse transcriptase throughout synthesizing.

A12. The method of any of paragraphs A1 to A11, wherein the cDNA accumulates in the reaction mixture at a substantially constant rate during synthesizing.

A13. The method of any of paragraphs A1 to A12, wherein synthesizing includes incubating the reaction mixture at a first temperature for a predefined time interval and then heating the reaction mixture to a second temperature at the end of the predefined time interval to stop synthesizing the cDNA.

A14. The method of paragraph A13, wherein the first temperature is above 25 degrees Celsius and the second temperature is above 70 degrees Celsius.

A15. The method of paragraph A14, wherein the first temperature is at least 35 degrees Celsius and the second temperature is at least 75 degrees Celsius.

A16. The method of any of paragraphs A1 to A15, wherein forming includes forming the partitions such that each partition includes a pair of primers to amplify the target.

A17. The method of any of paragraphs A1 to A16, wherein amplifying includes thermally cycling the partitions.

A18. The method of paragraph A17, wherein amplifying includes performing a polymerase chain reaction in the partitions.

A19. The method of any of paragraphs A1 to A18, wherein collecting amplification data includes detecting fluorescence from the partitions.

A20. The method of paragraph A19, wherein forming includes forming the partitions such that each partition includes a probe having a label, wherein the probe binds specifically to the target, and wherein detecting the fluorescence includes detecting the fluorescence from the label.

A21. The method of any of paragraphs A1 to A20, wherein forming the partitions includes forming droplets.

A22. The method of any of paragraphs A1 to A21, wherein the reverse transcriptase activity of the sample determines a rate of accumulation of the cDNA throughout synthesizing.

A23. A system for measuring reverse transcriptase activity of a sample, the system comprising: a plurality of partitions containing a cDNA at partial occupancy, each partition including a portion of a reaction mixture in which the cDNA was synthesized by reverse transcription using the reverse transcriptase activity of the sample and an RNA polymer as a template, the cDNA being present in an amount that is proportional to the reverse transcriptase activity of the sample, each partition also including reagents sufficient for amplification of a target representing the cDNA, if a copy of the cDNA is present in the partition.

A24. The system of paragraph A23, further comprising a holder to contain the plurality of partitions.

### V. Advantages and Benefits

The different examples of methods and systems for detecting reverse transcriptase activity provide several advantages over known solutions for detecting reverse transcriptase activity. For example, illustrative examples described herein permit reverse transcriptase activity to be measured without a reverse transcriptase standard, thereby avoiding inaccuracies and errors resulting from activity drift that can occur in such a standard.

Additionally, and among other benefits, illustrative examples disclosed herein permit quantification of reverse transcriptase activity of a sample without the use of a radioactive dNTP substrate.

Additionally, and among other benefits, illustrative examples disclosed herein enable absolute quantification of reverse transcriptase activity.

Additionally, and among other benefits, illustrative examples disclosed herein permit measurement of the activity of a reverse transcriptase by a manufacturer thereof.

Additionally, and among other benefits, illustrative examples disclosed herein permit measurement of the activity of a reverse transcriptase by an end user, to ensure that the reverse transcriptase has a desired, sufficient, or stated level of activity.

Additionally, and among other benefits, illustrative examples disclosed herein may be used to detect reverse transcriptase activity in cell culture supernatants or other biological samples. In some cases, the methods and systems may be used to detect and/or quantify retroviruses in cell supernatants or other biological samples.

No known method or system can perform these functions. However, not all examples described herein provide the same advantages or the same degree of advantage.

### VI. Conclusion

The disclosure set forth above may encompass multiple distinct examples with independent utility. Although each of these has been disclosed in its preferred form(s), the specific examples thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, processes, and/or properties disclosed herein. The following claims particularly point out certain combinations and subcombinations regarded as novel and nonobvious. Other combinations and subcombinations of elements, features, functions, processes, and/or properties may be claimed in applications claiming priority from this or a related application. Such claims, whether broader, narrower, equal, or different in scope to the original claims, also are regarded as included within the subject matter of the present disclosure.

We claim:

1. A method of detecting reverse transcriptase activity of a sample, the method comprising:
preparing a reaction mixture including the sample, an RNA polymer, and reagents for reverse transcription;
synthesizing complementary DNA (cDNA) in the reaction mixture using the RNA polymer as a template, wherein an amount of the cDNA synthesized is proportional to the reverse transcriptase activity of the sample;
forming partitions after synthesizing, the partitions containing copies of the cDNA at partial occupancy and each partition including a portion of the reaction mixture;
amplifying a target representing the cDNA in the partitions; and
collecting amplification data for the target from the partitions.

2. The method of claim 1, further comprising enumerating at least one population of the partitions defined according to positivity or negativity for the target using the amplification data.

3. The method of claim 2, further comprising enumerating the partitions without respect to positivity/negativity for the target to obtain a total number of the partitions.

4. The method of claim 2, further comprising determining a value for the reverse transcriptase activity of the sample based on enumerating.

5. The method of claim 4, wherein determining includes calculating a fraction of the partitions that is positive for the target or a fraction of the partitions that is negative for the target.

6. The method of claim 4, wherein determining includes calculating an average number of molecules of the cDNA per partition or unit volume.

7. The method of claim 1, wherein preparing the reaction mixture includes combining the sample and the RNA polymer with one another.

8. The method of any of claim 1, wherein the RNA polymer is a product of transcription in vitro.

9. The method of claim 1, wherein the sample includes at least one reverse transcriptase that produces the reverse transcriptase activity, and wherein the RNA polymer is present in at least a 20-fold, 50-fold, or 100-fold molar excess relative to the at least one reverse transcriptase after preparing the reaction mixture.

10. The method of claim 1, wherein the reagents for reverse transcription include deoxyribonucleoside triphosphates (dNTPs) and at least one primer for cDNA synthesis with the RNA polymer as template.

11. The method of claim 1, wherein the sample includes at least one reverse transcriptase that produces the reverse transcriptase activity, and wherein the RNA polymer is present at a non-limiting concentration with respect to the at least one reverse transcriptase throughout synthesizing.

12. The method of claim 1, wherein the cDNA accumulates in the reaction mixture at a substantially constant rate during synthesizing.

13. The method of claim 1, wherein synthesizing includes incubating the reaction mixture at a first temperature for a predefined time interval and then heating the reaction mixture to a second temperature at the end of the predefined time interval to stop synthesizing the cDNA.

14. The method of claim 1, wherein forming includes forming the partitions such that each partition includes a pair of primers to amplify the target.

15. The method of claim 1, wherein amplifying includes thermally cycling the partitions.

16. The method of claim 1, wherein collecting amplification data includes detecting fluorescence from the partitions.

17. The method of claim 16, wherein forming includes forming the partitions such that each partition includes a probe having a label, wherein the probe binds specifically to the target, and wherein detecting the fluorescence includes detecting the fluorescence from the label.

18. The method of claim 1, wherein forming the partitions includes forming droplets.

19. The method of claim 1, wherein the reverse transcriptase activity of the sample determines a rate of accumulation of the cDNA throughout synthesizing.

20. A system for measuring reverse transcriptase activity of a sample, the system comprising:
a plurality of partitions containing a cDNA at partial occupancy, each partition including a portion of a reaction mixture in which the cDNA was synthesized by reverse transcription using the reverse transcriptase activity of the sample and an RNA polymer as a template, the cDNA being present in an amount that is proportional to the reverse transcriptase activity of the sample, each partition also including reagents sufficient for amplification of a target representing the cDNA, if a copy of the cDNA is present in the partition.
